(19) United States
(12) Patent Application Publication
(10) Pub. No.: US 2004/0258654 A1
(43) Pub. Date: Dec. 23, 2004

(54) Cosmetic or dermatological formulations containing glycerin

(75) Inventors: Jens Nielsen, Hensstedt-Ulzburg (DE); Rainer Kropke, Schenefeld (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/832,837

(22) Filed: Apr. 26, 2004

Related U.S. Application Data
(63) Continuation of application No. PCT/EP02/11792, filed on Oct. 22, 2002.

(30) Foreign Application Priority Data
Oct. 26, 2001 (DE) 101 52 304.1

(51) Int. Cl. A61K 7/32; A61K 7/075; A61K 7/08

(52) U.S. Cl. 424/70.31

(57) ABSTRACT

The present invention is a cosmetic or dermatological topical formulation based on an oil-in-water emulsion, comprising (a) 0.05 wt. % to 2 wt. %, based on the total weight of the formulation, of one or more ethoxylated fatty acid esters selected from the group consisting of PEG-5 to PEG-100 stearates; (b) 0.5 wt. % to 20 wt. %, based on the total weight of the formulation, of glycerin; and (c) one or more of (i) 0.1 wt. % to 6 wt. %, based on the total weight of the formulation, of glycerol monostearate, and (ii) 0.1 wt. % to 8 wt. %, based on the total weight of the formulation, of one or more C₁₆-C₁₈ fatty alcohols. The present invention also includes a method of using the formulation to moisturize the skin and a method of using the formulation to limit aging of the skin, limit the formation of wrinkles, treat aged skin and/or treat wrinkles.

### CROSS-REFERENCE TO RELATED APPLICATIONS

This is a continuation application of PCT/EP02/11792 filed Oct. 22, 2002, which is incorporated herein by reference in its entirety, and also claims the benefit of German Priority Application No. 101 52 304.1, filed Oct. 26, 2001.

### FIELD OF THE INVENTION

The present invention relates to cosmetic and dermatological emulsions, in particular skin care cosmetic and dermatological emulsions. In an advantageous embodiment, the present invention relates to methods of increasing and improving the skin-moisturizing action of oil-in-water (O/W) emulsions. The present invention also relates to cosmetic or pharmaceutical formulations having a reduced feeling of stickiness, processes for their preparation and the use of active compounds for reducing the feeling of stickiness of cosmetic formulations.

### BACKGROUND OF THE INVENTION

The skin is the largest organ of the human body. Among its many functions (for example for heat regulation and as a sensory organ) is the barrier function, which prevents the skin (and therefore in the end the entire organism) from drying out, which is certainly the most important. At the same time the skin acts as a protective device against penetration and absorption of substances coming from the outside. This barrier function is affected by the epidermis, which as the outermost layer forms the actual protective covering against the environment. At about one tenth of the total thickness, it is at the same time the thinnest layer of the skin.

The epidermis is a stratified tissue in which the outer layer, the horny layer (stratum corneum) is the important part for the barrier function. The skin model of Elias acknowledged nowadays among experts (P. M. Elias, Structure and Function of the Stratum Corneum Permeability Barrier, Drug Dev. Res. 13, 1988, 97-105) describes the horny layer as a two-component system, similar to a brick wall (brick-mortar model). In this model, the horny cells (corneocytes) correspond to the bricks and the lipid membrane of complex composition in the intercellular spaces corresponds to the mortar. This system substantially represents a physical barrier against hydrophilic substances, but because of it close and multi-layered structure, lipophilic substances also can likewise pass through only with difficulty.

Apart from their barrier action against external chemical and physical influences, the epidermal lipids also contribute towards the cohesion of the horny layer and have an influence on the smoothness of the skin. In contrast to the lipids of the sebaceous glands, which do not form a closed film on the skin, the epidermal lipids are distributed over the entire horny layer.

The extremely complex interaction of the moisture-binding substances and the lipids of the upper layers of skin is very important for regulation of the skin moisture. Cosmetics therefore as a rule comprise water-binding substances or humectants, in addition to balanced lipid blends and water.

In addition to the chemical composition, however, the physical properties of these substances are also of importance. The development of emulsifiers or surfactants of very good biocompatibility is therefore desirable. Products formulated with these assist the liquid crystal organization of the intercellular lipids of the stratum corneum and in this way improve the barrier properties of the horny layer. It is particularly advantageous if their molecular constituents consist of substances which occur naturally in the epidermis.

Cosmetic skin care is to be understood primarily as meaning that the natural function of the skin as a barrier against environmental influences (e.g. dirt, chemicals and microorganisms) and against the loss of endogenous substances (e.g. water, naturally occurring fats and electrolytes) is intensified or re-established.

If this function is impaired, increased absorption of toxic or allergenic substances or attack by microorganisms and as a consequence toxic or allergic skin reactions may occur.

The aim of skin care is furthermore to compensate the loss of fats and water caused by daily washing. This is important precisely if the natural capacity for regeneration is inadequate. Skin care products should moreover protect against environmental influences, in particular against sun and wind, and delay aging of the skin.

Medical topical compositions as a rule comprise one or more medicaments in an active concentration. For simplicity, for a clear distinction between cosmetic and medical use and corresponding products reference is made to the legal provisions of the Federal Republic of Germany (e.g. cosmetics legislation, foodstuffs and medical preparations law).

Conventional cosmetic presentation forms are emulsions. These are in general understood as meaning a heterogeneous system of two liquids which are immiscible or of only limited miscibility with one another, which are conventionally called phases. One is present here in the form of droplets (disperse or internal phase), while the other liquid forms a continuous (coherent or internal) phase. Rarer presentation forms are multiple emulsions, that is to say those which in their turn contain in the droplets of the dispersed (or discontinuous) phase droplets of a further dispersed phase, e.g. W/O/W emulsions and O/W/O emulsions.

More recent findings have recently led to a better understanding of cosmetic emulsions which are relevant in practice. It is assumed here that the emulsifier mixtures employed in excess form lamellar liquid crystal phases or crystalline gel phases. In the gel network theory, the stability and physicochemical properties of such emulsions are attributed to the formation of viscoelastic gel networks.

To be able to ensure the metastability of emulsions, as a rule surface-active substances, that is to say emulsifiers, are necessary. In itself, the use of the conventional cosmetic emulsifiers is completely harmless. Nevertheless, emulsifiers, as any chemical substance in the end, cause allergic reactions or reactions based on the hypersensitivity of the user in the individual case. It is thus known that in some particularly sensitive people certain photodermatoses are induced by certain emulsifiers and the simultaneous action of sunlight.

It is possible to prepare emulsifier-free formulations which have, for example, oil droplets dispersed in an aqueous phase, similarly to an O/W emulsion. A prerequisite for this can be that the continuous aqueous phase has a gel matrix which stabilizes the dispersed phase and other further circumstances. Such systems are occasionally called hydrodispersions or oleodispersions, depending on which is the disperse and which is the continuous phase.

However, for cosmetics formulations, it is neither necessary nor possible to dispense with emulsifiers entirely, especially since a certain choice of particularly mild emulsifiers exists. However, there is a lack of prior art of a satisfactorily wide diversity of such emulsifiers, which would then also significantly broaden the use spectrum of correspondingly milder and more skin-tolerable cosmetic formulations.

### SUMMARY OF THE INVENTION

An object of the present invention was thus to provide cosmetic and dermatological formulations with outstanding skin care properties which in particular increase and improve moisturizing of the skin.

It was furthermore an object of the present invention to provide formulations which significantly improve the condition of the skin, in particular the roughness of the skin.

It is indeed known to reduce a feeling of stickiness or also a feeling of greasiness by addition of particular substances, for example some selected powder raw materials, in particular talc. Apart from the fact that this is only rarely completely successful, the viscosity of the product in question is also changed and the stability reduced by such an addition.

The object was therefore to provide a remedy to all these, the disadvantages of the prior art. In particular, products with a reduced stickiness or greasiness should be provided. Products in the field of care cosmetics, of decorative cosmetics and of pharmacological formulation should likewise be freed from the disadvantages described for the prior art.

It was moreover an object of the invention to develop cosmetic bases for cosmetic formulations which are distinguished by a good skin tolerability.

It was furthermore an object of the present invention to provide products with a broadest possible diversity of uses. For example, bases for formulation forms such as cleansing emulsions, face and body care formulations, and also decidedly medical-pharmaceutical presentation forms should be provided achieved.

The objects described are achieved according to the invention. The invention relates to cosmetic or dermatological topical formulations in the form of O/W emulsions with a content of 0.05 to 2 wt. %, based on the total weight of the formulations, of one or more ethoxylated fatty acid esters chosen from the group consisting of PEG-5 to PEG-100 stearates in combination with

(A) 0.1 wt. % to 6 wt. %, based on the total weight of the formulations, of glycerol monostearate, or in combination with

(B) 0.1 wt. % to 8 wt. %, based on the total weight of the formulations, of one or more C₁₆-C₁₈ fatty alcohols, or

(C) in combination with 0.1 wt. % to 6 wt. %, based on the total weight of the formulations, of glycerol monostearate and 0.1 wt. % to 8 wt. %, based on the total weight of the formulations, of one or more C₁₆-C₁₈ fatty alcohols, and

furthermore with a content of 0.5 wt. % to 20 wt. %, based on the total weight of the formulations, of glycerin and 0 wt. %, in particular 0.1 wt. % to 30 wt. %, based on the total weight of the formulations, of one or more lipids having a polarity index of 5-30 mN/m, in particular 10-25 mN/m, this index range also applying to mixtures of lipids, and water and, optionally, active compounds, auxiliaries and/or additives.

Glycerol monostearate (GMS, alpha-monostearin or also called glyceryl stearate here) is known, is described in the literature and preferably contains no or only low, i.e. of industrial origin, contents of triglycerides or fatty acids.

Preferred ethoxylated fatty acid esters are chosen from the group consisting of PEG-20 to PEG-60 stearates. Ethoxylated fatty acid esters are preferably contained in amounts of 0.2 to 1 wt. %, in each case based on the total weight of the formulation.

Glycerol monostearate is preferably contained in amounts of 0.5 to 3 wt. %, in each case based on the total weight of the formulation.

Fatty alcohols are preferably contained in amounts of 0.5 to 4 wt. %, in each case based on the total weight of the formulation.

Cosmetic or dermatological topical formulations in the form of O/w emulsions with a content of 0.2 to 1 wt. %, based on the total weight of the formulations, of one or more ethoxylated fatty acid esters chosen from the group consisting of PEG-20 to PEG-60 stearates in combination with

(A) 0.5 wt. % to 3 wt. %, based on the total weight of the formulations, of glycerol monostearate, or in combination with

(B) 0.5 wt. % to 4 wt. %, based on the total weight of the formulations, of one or more C₁₆-C₁₈ fatty alcohols, or

(C) in combination with 0.5 wt. % to 3 wt. %, based on the total weight of the formulations, of glycerol monostearate and 0.5 wt. % to 4 wt. %, based on the total weight of the formulations, of one or more C₁₆-C₁₈ fatty alcohols,

and furthermore with a content of 1 wt. % to 10 wt. %, based on the total weight of the formulations, of glycerin and 0 wt. %, in particular 0.5 wt. % to 20 wt. %, based on the total weight of the formulations, of one or more lipids having a polarity index of 5-30 mN/m, in particular 10-25 mN/m, this index range also applying to mixtures of lipids, and water and, optionally, active compounds, auxiliaries and/or additives, are preferred.

The invention also relates to the use of topical formulations in the form of O/W emulsions with a content of 0.05 to 2 wt. %, based on the total weight of the formulations, of one or more ethoxylated fatty acid esters chosen from the group consisting of PEG-5 to PEG-100 stearates in combination with

(A) 0.1 wt. % to 6 wt. %, based on the total weight of the formulations, of glycerol monostearate, or in combination with

(B) 0.1 wt. % to 8 wt. %, based on the total weight of the formulations, of one or more C₁₆-C₁₈ fatty alcohols, or

(C) in combination with 0.1 wt. % to 6 wt. %, based on the total weight of the formulations, of glycerol monostearate and 0.1 wt. % to 8 wt. %, based on the total weight of the formulations, of one or more C₁₆-C₁₈ fatty alcohols,

furthermore with a content of 0.5 wt. % to 20 wt. %, based on the total weight of the formulations, of glycerin and 0 wt. %, in particular 0.1 wt. % to 30 wt. %, based on the total weight of the formulations, of one or more lipids having a polarity index of 5-30 mN/m, in particular 10-25 mN/m, this index range also applying to mixtures of lipids, and water and, optionally, active compounds, auxiliaries and/or additives, for improving moisturizing of the skin.

The use of topical formulations in the form of O/W emulsions with a content of 0.2 to 1 wt. %, based on the total weight of the formulations, of one or more ethoxylated fatty acid esters chosen from the group consisting of PEG-20 to PEG-60 stearates in combination with

(A) 0.5 wt. % to 3 wt. %, based on the total weight of the formulations, of glycerol monostearate, or in combination with

(B) 0.5 wt. % to 4 wt. %, based on the total weight of the formulations, of one or more C₁₆-C₁₈ fatty alcohols, or

(C) in combination with 0.5 wt. % to 3 wt. %, based on the total weight of the formulations, of glycerol monostearate and 0.5 wt. % to 4 wt. %, based on the total weight of the formulations, of one or more C₁₆-C₁₈ fatty alcohols, and

furthermore with a content of 1 wt. % to 10 wt. %, based on the total weight of the formulations, of glycerin and 0 wt. %, in particular 0.5 wt. % to 20 wt. %, based on the total weight of the formulations, of one or more lipids having a polarity index of 5-30 mN/m, in particular 10-25 mN/m, this index range also applying to mixtures of lipids, and water and, optionally, active compounds, auxiliaries and/or additives, for moisturizing the skin is preferred.

It had therefore not been foreseeable to the expert that the formulations according to the invention would

have a better action than moisturizing formulations,

better promote smoothing of the skin,

be distinguished by a better care action,

serve better as a vehicle for cosmetic and medical-dermatological active compounds, and

be distinguished by a better feeling on the skin and by a higher cosmetic elegance,

than the formulations of the prior art.

### DETAILED DESCRIPTION OF THE PREFERRED EMBODIMENTS

The formulations according to the invention can be formulated to be both flowable and cream-like, have very good cosmetic properties, in particular as regards the stickiness and the moisturizing of the skin, and have a very good skin tolerability and skin care performance.

Emulsions of the oil-in-water type conventionally comprise humectants which are chiefly responsible for the skin-moisturizing effect of this emulsion. When used in relatively high concentrations, however, humectants such as glycerin leave behind an unpleasant sticky feeling on the skin. The use of ethoxylated hydrophilic emulsifiers with the combination partners, in particular lipophilic emulsifiers which are not self-emulsifying, render possible, e.g., a reduction in the amount of the humectant glycerin and therefore the stickiness for the same moisturizing of the skin compared with O/W emulsions of the prior art.

In skin moisturizing tests the formulations according to the invention show a significant superiority over formulations of the prior art, which were provided with significantly more humectant, in respect of the skin moisture and the troublesome feeling of stickiness.

A further advantage of the formulations according to the invention is the low content of emulsifiers and the good skin tolerability which is achieved in particular in this manner.

Formulations which comprise no free fatty acids or salts thereof are preferred.

Formulations of variant C with a content of the combination comprising three components are particularly preferred.

Lipid-free formulations according to the invention preferably comprise one or more fatty alcohols, e.g. as stated in each case in B and C, or constituents with a lipid character, such as e.g. emulsifiers, for example in each case in amounts of 0.1 wt. % to 30 wt. %, preferably 0.5 to 20 wt. %, in each case based on the total weight of the formulations.

The substances or mixtures thereof preferably also have polarities in the stated index ranges of the lipids.

The moisturizing of the skin can be improved in respect of two effects in this manner: The moisturizing is increased according to the invention and/or the sensorially unpleasant stickiness is reduced or avoided completely according to the invention. In the context of the present disclosure, the term “lipids” is used as a generic term for fats, oils, waxes and the like, as is entirely familiar to the expert. The terms “oily phase” and “lipid phase” are also used synonymously.

The polarity index states the polarity or the surface tension (in 10⁻³ Newton/meter), which is determined with the ring method in a known manner. The measurements are carried out with a ring tensiometer at 20° C. against air.

Polar or medium-polarity lipids or lipid mixtures, which can also comprise contents of non-polar lipids, in which case the required polarity index range must be remembered, are preferred.

Polar oils are, for example, those from the group consisting of lecithins and fatty acid triglycerides, namely the triglycerol esters of saturated or unsaturated, branched or unbranched alkanecarboxylic acids of a chain length of 8 to 24, in particular 12 to 18 C atoms. The fatty acid triglycerides can advantageously be chosen, for example, from the group consisting of synthetic, semi-synthetic and naturally occurring oils, such as e.g. olive oil, sunflower oil, soya oil, groundnut oil, rape oil, almond oil, palm oil, coconut oil, castor oil, wheat germ oil, grape seed oil, thistle oil, evening primrose oil, macadamia nut oil and the like.

Further polar oil components can be chosen from the group consisting of esters of saturated or unsaturated, branched or unbranched alkanecarboxylic acids of a chain length of 3 to 30 C atoms and saturated or unsaturated, branched or unbranched alcohols of a chain length of 3 to 30 C atoms, as well as from the group consisting of esters of aromatic carboxylic acids and saturated or unsaturated, branched or unbranched alcohols of a chain length of 3 to 30 C atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate and synthetic, semi-synthetic and naturally occurring mixtures of such esters, such as e.g. jojoba oil.

The oily phase can furthermore advantageously be chosen from the group consisting of dialkyl ethers, the group consisting of saturated or unsaturated, branched or unbranched alcohols. It is advantageous in particular if the oily phase of the W/O emulsions according to the invention has a content of C₁₂-C₁₅-alkyl benzoate or consists entirely of this.

Any desired blends of such oil and wax components are also advantageously to be employed in the context of the present invention. It may also be advantageous, where appropriate, to employ waxes, for example cetyl palmitate, as the sole lipid component of the oily phase.

Non-polar oils which can also be contained in the lipid phase are, for example, those which are chosen from the group consisting of branched and unbranched hydrocarbons and -waxes, in particular vaseline (petrolatum) paraffin oil, squalane and squalene, polyolefins and hydrogenated polyisobutenes. Among the polyolefins, polydecenes are the preferred substances.

The following tables 1 and 2 list lipids which are advantageous according to the invention as individual substances or also in a mixture with one another. The relevant interfacial tensions against air are stated in the last column. However, it is also advantageous to use mixtures of more highly polar and/or low-polarity and non-polar lipids, as long as it is ensured that the total polarity of the oily phase is in the range claimed.

TABLE 1

| | | Polarity |
|---|---|---|
| | | index |
| Trade name | INCI name | (mN/m) |
| Isofol ® 14 T | Butyl Decanol + Hexyl Decanol + Hexyl | 27.6 |
| | Octanol + Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Miglyol ® 812 | Caprylic/Capric Triglyceride | 21.3 |
| Cegesoft ® C24 | Octyl Palmitate | 23.1 |
| Isopropyl stearate | Isopropyl Stearate | 21.9 |
| Estol ® 1540 EHC | Octyl Octanoate | 30.0 |
| Finsolv ® TN | C₁₂₋₁₅ Alkyl Benzoate | 21.8 |
| Cetiol ® SN | Cetearyl Isonoanoate | 28.6 |
| Dermofeel ® BGC | Butylene Glycol Caprylate/Caprate | 21.5 |
| Trivent ® OCG | Tricaprylin | 20.2 |
| MOD | Octyldodeceyl Myristate | 22.1 |
| Cosmacol ® ETI | Di-C₁₂₋₁₃ Alkyl Tartrate | 29.4 |
| Miglyol ® 829 | Caprylic/Capric Diglyceryl Succinate | 29.5 |
| Prisorine ® 2036 | Octyl Isostearate | 29.7 |
| Tegosoft ® SH | Stearyl Heptanoate | 28.7 |
| Abil ® Wax 9840 | Cetyl Dimethicone | 25.1 |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

TABLE 2

| INCI name | Polarity index (mN/m) |
|---|---|
| Mineral Oil | 43.7 |
| Mineral Oil | 43.7 |
| Mineral Oil | 38.3 |
| Isohexadecane | 43.8 |
| Cocoglycerides | 5.1 |
| Caprylic/Capric Triglycerides | 21.3 |
| Isoeicosane | 41.9 |
| Octyl Palmitate | 23.1 |
| Octyldodecanol | 24.8 |
| Isopropyl Stearate | 21.9 |
| Octyl Cocoate | 30.0 |
| C12-15 Alkyl Benzoate | 21.8 |
| Polydecene | 46.7 |
| Polydecene | 30.1 |
| Dicaprylyl Ether | 30.9 |
| Cetearyl Isononanoate | 28.6 |
| Propylene Glycol Dicaprylate/Dicaprate | 18.7 |
| Isopropyl Palmitate | 28.8 |
| Dibutyl Adipate | 14.3 |
| Dimethicone | 26.9 |
| Cyclomethicone | 28.5 |
| Phenyl Trimethicone | 22.7 |
| Dicaprylyl Carbonate | 31.7 |
| Butylene Glycol Caprylate/Caprate | 21.5 |
| Octyl Isostearate | 31.6 |
| Stearyl Heptanoate | 17.8 |
| Triisostearin | 2.4 |
| Tridecyl Stearate(+) Tridecyl Trimellitate(+), | 19.4 |
| Dipentaerythrityl | |
| Hexacaprylate/Hexacaprate | |
| Octyldodecyl Myristate | 22.1 |
| Pentaerythrityl Tetraisostearate | 3.1 |
| Persea Gratissima | 14.5 |
| Macadamia Ternifolia | 22.1 |
| Ricinus Communis | 19.2 |
| Buxus Chinensis | 26.2 |
| Hydrogenated Polyisobutene | 44.7 |
| Cyclomethicone | 28.5 |

The following can be used as base constituents of the formulations according to the invention:

water or aqueous solutions;

aqueous ethanolic solutions;

naturally occurring oils and/or chemically modified naturally occurring oils and/or synthetic oils;

fats, waxes and other naturally occurring and synthetic fat substances, preferably esters of fatty acids with alcohols of low C number, e.g. with isopropanol, propylene glycol or glycerin, or esters of fatty alcohols with alkanoic acids of low C number or with fatty acids; and

alcohols, diols or polyols of low C number, as well as ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

According to the invention, the oily phase of the emulsions in the context of the present invention preferably consists completely of medium-polarity or polar lipid components, although it is possible, without suffering great disadvantages, to choose up to 50 wt. %, preferably up to 40 wt. % of the total weight of the oil components from the group consisting of other oil components.

Lipids which are preferred according to the invention can advantageously be chosen from the group consisting of esters of saturated or unsaturated, branched or unbranched alkanecarboxylic acids of a chain length of 3 to 30 C atoms and saturated or unsaturated, branched or unbranched alcohols of a chain length of 3 to 30 C atoms, from the group consisting of esters of aromatic carboxylic acids and saturated or unsaturated, branched or unbranched alcohols of a chain length of 3 to 30 C atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and naturally occurring mixtures of such esters, e.g. jojoba oil, as long as the conditions required in the main claim are adhered to.

The oily phase furthermore can advantageously be chosen from the group consisting of branched and unbranched hydrocarbons and waxes, dialkyl ethers, the group consisting of saturated or unsaturated, branched or unbranched alcohols, as well as fatty acid triglycerides, namely the triglycerol esters of saturated or unsaturated, branched or unbranched alkanecarboxylic acids of a chain length of 8 to 24, in particular 12-18 C atoms. The fatty acid triglycerides can advantageously be chosen, for example, from the group consisting of synthetic, semi-synthetic and naturally occurring oils, e.g. olive oil, sunflower oil, soya oil, groundnut oil, rape oil, almond oil, palm oil, coconut oil, palm kernel oil and many of the like, as long as the conditions required in the main claim are adhered to.

Fat or wax components which are advantageously to be used according to the invention can be chosen from the group consisting of plant waxes, animal waxes, mineral waxes and petrochemical waxes. Those which are favorable according to the invention are, for example, candelilla wax, carnauba wax, Japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygium wax, ceresin, ozocerite (earth wax), paraffin waxes and microwaxes, as long as the conditions required in the main claim are adhered to.

Further advantageous fat or wax components are chemically modified waxes and synthetic waxes, such as, for example, those obtainable under the trade names Syncrowax HRC (glyceryl tribehenate), Syncrowax HGLC (C₁₆₋₃₆-fatty acid triglyceride) and Syncrowax AW 1C(C₁₈₋₃₆-fatty acid) from CRODA GmbH as well as montan ester waxes, Sasol waxes, hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, and also chemically modified fats, such as e.g. hydrogenated plant oils (for example hydrogenated castor oil and/or hydrogenated coconut fatty glycerides), triglycerides, such as, for example, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Certain organosilicon compounds which have similar physical properties to the fat or wax components mentioned, such as, for example, stearoxytrimethylsilane, are also furthermore advantageous, as long as the conditions required in the main claim are adhered to.

According to the invention, the fat or wax components can be present both individually and in a mixture. Any desired blends of such oil and wax components are also advantageously to be employed in the context of the present invention. It may also be advantageous, where appropriate, to employ waxes, for example cetyl palmitate, as the sole lipid component of the oily phase, as long as the conditions required in the main claim are adhered to.

The oily phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, capryl-capric acid triglyceride, dicaprylyl ether, as long as the conditions required in the main claim are adhered to.

Mixtures of C₁₂₋₁₅-alkyl benzoate and 2-ethylhexyl isostearate, mixtures of C₁₂₋₁₅-alky benzoate and isotridecyl isononanoate and mixture of C₁₂₋₁₅ alky benzoate, 2-ethylhexyl isostearate and isotridecyl isononanoate are particularly advantageous, as long as the conditions required in the main claim are adhered to.

If desired, O/W emulsions according to the invention can also comprise conventional O/W emulsifiers, if desired also W/O emulsifiers and further coemulsifiers.

If desired, O/W emulsions corresponding to the present invention can comprise one or more emulsifiers, in particular advantageously chosen from the group consisting of the following substances, which as a rule act as W/O emulsifiers:

Sorbitan stearate, sorbitan oleate, lecithin, glyceryl lanolate, lanolin, microcrystalline wax (cera microcristallina) in a mixture with paraffin oil (paraffinum liquidum), ozocerite, hydrogenated castor oil, glyceryl isostearate, polygylceryl-3 oleate, wool wax acid mixtures, wool wax alcohol mixtures, pentaerythrithyl isostearate, polyglyceryl-3 diisostearate, sorbitan oleate in a mixture with hydrogenated castor oil, beeswax (cera alba) and stearic acid, sodium dihydroxycetyl phosphate in a mixture with isopropyl hydroxycetyl ether, methylglucose dioleate, methylglucose dioleate in a mixture with hydroxystearate and beeswax, mineral oil in a mixture with petrolatum and ozocerite and glyceryl oleate and lanolin alcohol, petrolatum in a mixture with ozocerite and hydrogenated castor oil and glyceryl isostearate and polyglyceryl-3 oleate, PEG-7 hydrogenated castor oil, sorbitan oleate in a mixture with PEG-2 hydrogenated castor oil, ozocerite and hydrogenated castor oil, sorbitan isostearate in a mixture with PEG-2 hydrogenated castor oil, polyglyceryl-4 isostearate, polyglyceryl-4 isostearate in a mixture with cetyldimethicone copolyol and hexyl laurate, laurylmethicone copolyol, cetyldimethicone copolyol, acrylate/C₁₀₋₃₀-alkyl acrylate cross-polymer, sorbitan isostearate, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, polyglyceryl-2 dipolyhydroxystearate, polyglyceryl-3 dipolyhydroxystearate, polyglyceryl-4 dipolyhydroxystearate and polyglyceryl-3 dioleate.

If desired, O/W emulsions corresponding to the present invention can comprise one or more emulsifiers, in particular advantageously chosen from the group consisting of the following substances, which as a rule act as O/W emulsifiers glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, glyceryl stearate, sodium cetylstearyl sulfate, lecithin, trilaureth-4 phosphate, laureth-4 phosphate, stearic acid, propylene glycol stearate SE, PEG-25-hydrogenated castor oil, PEG-54 hydrogenated castor oil, PEG-6 caprylic acid/capric acid glycerides, glyceryl oleate in a mixture with propylene glycol, PEG-9 stearate, ceteth-2, ceteth-20, polysorbate 60, glyceryl stearate in a mixture with PEG-100 stearate, glyceryl myristate, glyceryl laurate, PEG-40 sorbitan peroleate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, methylglucose sesquistearate, steareth-10, PEG-20 stearate, steareth-2 in a mixture with PEG-8 distearate, steareth-21, steareth-20, isosteareth-20, PEG-45/dodecyl glycol copolymer, methoxy-PEG-22/dodecyl glycol copolymer, PEG-40 sorbitan peroleate, PEG-40 sorbitan perisostearate, PEG-20 glyceryl stearate, PEG-20 glyceryl stearate, PEG-8 beeswax, polyglyceryl-2 laurate, isostearyl diglycerylsuccinate, stearamidopropyl-PG-dimonium chloride phosphate, glyceryl stearate SE, ceteth-20, triethyl citrate, PEG-20 methylglucose sesquistearate, cetaereth-12, glyceryl stearate citrate, cetyl phosphate, sorbitan sesquioleate, triceteareth-4 phosphate, trilaureth-4 phosphate, polyglyceryl methylglucose distearate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, cetylstearyl alcohol and cetyl palmitate, cetylstearyl alcohol in a mixture with PEG-20 stearate, PEG-30 stearate, PEG-40 stearate and PEG-100 stearate.

The above emulsifiers can be contained in the formulations, for example, in amounts of 0.01 to 10 wt. %, preferably 0.1 to 5 wt. % and in particular 0.5 to 3 wt. %, in each case based on the total weight of the formulations, as long as the conditions required in the main claim are adhered to.

Emulsion according to the invention in the context of the present invention, e.g. in the form of a skin protection cream, a hand lotion, a cosmetic milk, for example in the form of a sun protection cream or a sun protection milk, are advantageous and comprise e.g. fats, oils, waxes and/or other fat substances, as well as water and one or more emulsifiers such as are conventionally used for such a type of formulation.

It is of course known to the expert that demanding cosmetic compositions usually are inconceivable without the conventional auxiliaries and additives. These include, for example, agents which impart consistency, fillers, perfume, dyestuffs, emulsifiers, additional active compounds, such as vitamins or proteins, light stabilizers, stabilizers, insect repellants, alcohol, water, salts, antimicrobially, proteolytically or keratolytically active substances etc.

The pH of the formulations according to the invention can be in the conventional range for cosmetics, e.g. pH 4.5-7.5, preferably 5.5-6.5, in particular pH 6.

Mutatis mutandis, corresponding requirements apply to the formulation of medical formulations.

Medical topical compositions in the context of the present invention as a rule comprise one or more medicaments in an active concentration. For simplicity, for a clear distinction between cosmetic and medical use and corresponding products reference is made to the legal provisions of the Federal Republic of Germany (e.g. cosmetics legislation, foodstuffs and medical preparations law).

Cosmetic or topical dermatological compositions in the context of the present invention can accordingly be used, for example, as skin protection cream, cleansing milk, sun protection lotion, nutrient cream, day or night cream etc., depending on their build-up. It is possible and advantageous, where appropriate, to use the compositions according to the invention as a base for pharmaceutical formulations.

It is also of advantage to use the properties according to the invention in the form of decorative cosmetics (make-up formulations).

Those cosmetic and dermatological formulations which are in the form of a sun protection composition are also favorable. These preferably additionally comprise, in addition to the active compound used according to the invention, at least one UVA filter substance and/or at least one UVB filter substance and/or at least one inorganic pigment.

However, it is also advantageous in the context of the present invention to formulate those cosmetic and dermatological formulations of which the chief purpose is not protection from sunlight, but which nevertheless comprise a content of UV protection substances. Thus, for example, UV-A or UV-B filter substances are usually incorporated into day creams.

Formulations according to the invention can advantageously comprise substances which absorb UV radiation in the UVB range, the total amount of the filter substances being e.g. 0.1 wt. % to 30 wt. %, preferably 0.5 to 10 wt. %, in particular 1 to 6 wt. %, based on the total weight of the formulations.

The UVB filters can be oil-soluble or water-soluble. Oil-soluble substances which may be mentioned are e.g.

3-benzylidienecamphor and derivatives thereof, e.g. 3-(4-methylbenzylidene)camphor,

4-aminobenzoic acid derivatives, preferably 4-(dimethylamino)benzoic acid (2-ethylhexyl) ester, 4-(dimethylamino)benzoic acid amyl ester;

esters of cinnamic acid, preferably 4-methoxycinnamic acid (2-ethylhexyl) ester, 4-methoxycinnamic acid isopentyl ester;

esters of salicylic acid, preferably salicylic acid (2-ethylhexyl) ester, salicylic acid (4-isopropylbenzyl) ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di(2-ethylhexyl) ester; and

2,4,6-trianilino-(p-carbo-2′-ethyl-1′-hexyloxy)-1,3,5-triazine

Advantageous water-soluble substances are:

2-phenylbenzimidazole-5-sulfonic acid and salts thereof, e.g. sodium, potassium or triethanolammonium salts,

sulfonic acid derivatives of benzophenones, preferably 2-hydroxy-4-methoxybenzophenone-5-sulfonic acid and its salts; and

sulfonic acid derivatives of 3-benzylidenecamphor, such as e.g. 4-(2-oxo-3-bornylidenemethyl)benzenesulfonic acid, 2-methyl-5-(2-oxo-3-bornylidenemethyl)sulfonic acid and their salts.

The list of the UVB filters mentioned which can be used according to the invention is of course not intended to be limiting.

It may also be of advantage to employ in formulations according to the invention UVA filters which are conventionally contained in cosmetic and/or dermatological formulations. Such filter substances are preferably derivatives of dibenzoylmethane, in particular 1-(4′-tert-butylphenyl)-3-(4′-methoxyphenyl)-propane-1,3-dione and 1-phenyl-3-(4′-isopropylphenyl)propane-1,3-dione. The invention also relates to formulations which comprise these combinations. The same amounts of UVA filter substances which have been mentioned for UVB filter substances can be used.

Cosmetic or dermatological formulations in the context of the present invention can also comprise inorganic pigments which are conventionally used in cosmetics to protect the skin from UV rays. These are oxides of titanium, zinc, iron, zirconium, silicon, manganese, aluminum, cerium and mixtures thereof, as well as modifications in which the oxides are the active agents. They are particularly preferably pigments based on titanium dioxide. The amounts mentioned for the above-mentioned combinations can be used.

The cosmetic and dermatological formulations according to the invention can comprise cosmetic active compounds, auxiliaries and/or additives such as are conventionally used in such formulations, e.g. antioxidants, preservatives, bactericides, perfumes, substances for preventing foaming, dyestuffs, pigments which have a coloring action, thickeners, surface-active substances, emulsifiers, softening, moistening and/or humectant substances, fats, oils, waxes or other conventional constituents of a cosmetic or dermatological formulation, such as alcohols, polyols, polymers, foam stabilizers, electrolytes organic solvents or silicone derivatives.

It is advantageous to add further anti-irritant or anti-inflammatory active compounds to the formulations in the context of the present invention, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol.

It is also advantageous to add conventional antioxidants to the formulations in the context of the present invention. According to the invention, all antioxidants which are suitable or customary for cosmetic and dermatological uses can be used as favorable antioxidants.

The antioxidants are advantageously chosen from the group consisting of amino acids (e.g. glycine, histidine, tyrosine, tryptophan) and derivatives thereof, imidazoles (e.g. urocaninic acid) and derivatives thereof, peptides, such as D,L-carnosine, D-carnosine, L-carnosine and derivatives thereof (e.g. anserine), carotenoids, carotenes (e.g. α-carotene, β-carotene, ψ-lycopine) and derivatives thereof, chlorogenic acid and derivatives thereof, liponic acid and derivatives thereof (e.g. dihydroliponic acid), aurothioglucose, propylthiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximes, homocysteine sulfoxime, buthionine sulfones, penta- hexa-heptathionine sulfoxime) in very small tolerable dosages (e.g. pmol to μmol/kg), furthermore (metal) chelating agents (e.g. α-hydroxy-fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferylbenzoate of benzoin resin, rutic acid and derivatives thereof, α-glycosylrutin, ferulic acid, furfurylideneglucitol, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄) selenium and derivatives thereof (e.g. selenium-methionine), stilbenes and derivatives thereof (e.g. stilbene oxide, trans-stilbene oxide) and the derivatives (salts, esters, ethers, sugars, nucleotides, nucleosides, peptides and lipids), which are suitable according to the invention, of these active compounds mentioned.

The amount of the antioxidants (one or more compounds) in the formulations is preferably 0.001 to 30 wt. %, particularly preferably 0.05-20 wt. %, in particular 1-10 wt. %, based on the total weight of the formulation.

If vitamin E and/or derivatives thereof are the antioxidant or antioxidants, it is advantageous to choose the particular concentrations thereof from the range of 0.001-10 wt. %, based on the total weight of the formulation.

Formulations according to the present invention can also be used as the base for cosmetic or dermatological deodorants or antiperspirants. All the active compounds which are usual for deodorants or antiperspirants can advantageously be used, for example odor maskers, such as the usual perfume constituents, odor absorbers, for example the laminar silicates described in the patent laid-open specification DE-P 40 09 347, of these in particular montmorillonite, kaolinite, ilite, beidellite, nontronite, saponite, hectorite, bentonite, smectite, furthermore, for example, zinc salts of ricinoleic acid.

Germ-inhibiting agents are also suitable for incorporation into the formulations according to the invention. Advantageous substances are, for example, 2,4,4′-trichloro-2′-hydroxydiphenyl ether (Irgasan) 1,6-di-(4-chlorophenylbiguanido)-hexane (chlorhexidine), 3,4,4′-trichlorocarbanilide, quaternary ammonium compounds, clove oil, mint oil, thyme oil, triethyl citrate, farnesol (3,7,11-trimethyl-2,6,10-dodecatrien-1-ol) and the active compounds and active compound combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium bicarbonate is also advantageously to be used.

The amount of such active compounds (one or more compounds) in the formulations according to the invention is preferably 0.001 to 30 wt. %, particularly preferably 0.05-20 wt. %, in particular 1-10 wt. %, based on the total weight of the formulation.

The aqueous phase of the cosmetic formulations in the context of the present invention can also have a gel character which, in addition to an active content of substances employed according to the invention and the solvents conventionally used for these, preferably water, also comprise further organic thickeners, e.g. gum arabic, xanthan gum, sodium alginate, starch and starch derivatives (e.g. di-starch phosphate), cellulose, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, organically modified or also non-modified hectorites, bentonites, or the like, or a mixture of polyethylene glycol and polyethylene glycol stearate or distearate. The thickener is contained in the gel e.g. in an amount of between 0.1 and 30 wt. %, preferably between 0.5 and 15 wt. %.

It may furthermore be of advantage to add interface- or surface-active agents, for example cationic emulsifiers, such as, in particular, quaternary surfactants, to the formulations according to the invention.

Quaternary surfactants contain at least one N atom which is covalently bonded with 4 alkyl or aryl groups. This leads to a positive charge, independently of the pH. Alkylbetaine, alkylamidopropylbetaine and alkylamidopropylhydroxysulfaine are advantageous. The cationic surfactants used according to the invention can furthermore preferably be chosen from the group consisting of quaternary ammonium compounds, in particular benzyltrialkylammonium chlorides or bromides, such as, for example, benzyldimethylstearylammonium chloride, and furthermore alkyltrialkylammonium salts, for example for example cetyltrimethylammonium chloride or bromide, alkyldimethylhydroxyethylammonium chlorides or bromides, dialkyldimethylammonium chlorides or bromides, alkylamidethyltrimethylammonium ether-sulfates, alkylpyridinium salts, for example lauryl- or cetylpyrimidinium chloride, imidazoline derivatives and compounds with a cationic character, such as amine oxides, for example alkyldimethylamine oxides or alkylaminoethyldimethylamine oxides. Cetyltrimethylammonium salts in particular are advantageously to be used.

It is also advantageous to employ cationic polymers (e.g. Jaguar® C 162 [Hydroxypropyl Guar Hydroxypropyltrimonium Chloride]) and modified magnesium aluminum silicates (e.g. Quaternium-18-Hectorite, which is obtainable e.g. under the trade name Bentone® 38 from Rheox, or Stearalkonium Hectorite, which is obtainable e.g. under the trade name Softisan® Gel from Hüls AG).

Formulations according to the invention can also advantageously comprise oil thickeners in order to improve the tactile properties of the emulsion and the stick consistency. Advantageous oil thickeners in the context of the present invention are, for example, further solids, such as e.g. hydrophobic silicon oxides of the Aerosil type, which are obtainable from Degussa AG. Advantageous Aerosil® types are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and Aerosil® R976.

So-called metal soaps (i.e. the salts of higher fatty acids with the exception of the alkali metal salts) are furthermore also advantageous oil thickeners in the context of the present invention, such as, for example, aluminum stearate, zinc stearate and/or magnesium stearate.

It is also advantageous to add amphoteric or zwitterionic surfactants (e.g. cocoamidopropylbetaine) and moisturizers (e.g. betaine) to formulations according to the invention. Amphoteric surfactants which are advantageously to be used are, for example, acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of the surface- or interface-active substances (one or more compounds) in the formulations according to the invention is preferably 0.001 to 30 wt. %, particularly preferably 0.05-20 wt. %, in particular 1-10 wt. %, based on the total weight of the formulation.

Formulations according to the invention can also comprise active compounds (one or more compounds) which are chosen from the group consisting of: acetylsalicylic acid, atropine, azulene, hydrocortisone and derivatives thereof, e.g. hydrocortisone 17-valerate, vitamins, e.g. ascorbic acid and derivatives thereof, vitamins of the B and D series, very favorably vitamin B₁, vitamin B₁₂ vitamin D₁, and also bisabolol, unsaturated fatty acids, namely the essential fatty acids (often also called vitamin F), in particular γ-linolenic acid, oleic acid, eicosapentaenoic acid, docosahexaenoic acid and derivatives thereof, chloramphenicol, caffeine, prostaglandins, thymol, camphor, extracts or other products of plant and animal origin, e.g. evening primrose oil, borage oil or currant kernel oil, fish oils, cod-liver oil and also ceramides and ceramide-like compounds and so on. It is also advantageous to choose the active compounds from the group consisting of the re-oiling substances, for example purcellin oil, Eucerit® and Neocerit®.

Preferred active compounds (one or more compounds) are ubiquinones, preferably coenzyme Q7-11, in particular coenzyme Q10, retinyl palmitate, tocopheryl acetate, flavones and/or flavonoids, in particular alpha-glucosylrutin, ascorbic acid, liponic acid and retinol. Coenzyme Q10 and/or alpha-glucosylrutin and formulations according to the invention which comprise these are particularly preferred. Formulations according to the invention which comprise coenzyme Q10 and/or retinol are also particularly preferred.

Formulations according to the invention with a content of the above-mentioned active compounds (one or more compounds) and combinations thereof are used in particular against aging of the skin and the formation of wrinkles and for treatment of wrinkles and aged skin.

The amount of such active compounds (one or more compounds) in the formulations according to the invention is preferably 0.001 to 30 wt. %, particularly preferably 0.01-20 wt. %, in particular 1-10 wt. %, based on the total weight of the formulation.

The amount of the ubiquinones (one or more compounds), in particular coenzyme Q10, in the formulations according to the invention is preferably 0.001 to 1 wt. %, particularly preferably 0.01 to 0.3 wt. %.

The amount of the flavones or flavonoids (one or more compounds) in the formulations, in particular alpha-glucosylrutin, is preferably 0.01 to 1 wt. %.

Unless stated otherwise, all the amounts data, percentages data or parts relate to the weight, in particular the total weight of the formulations or of the particular mixture.

The following examples are intended to illustrate the present invention.

All data in wt. %.

| Example | | | | | | | | |
|---|---|---|---|---|---|---|---|---|
| number | 1 | 2 | 3 | 4 | 5 | 6 | 7 | 8 |
| PEG-10 xstear- | 0.5 | 1 | | | | | | 1.5 |
| ate | | | | | | | | |
| PEG-40 stear- | | | 1 | | | 1.5 | | |
| ate | | | | | | | | |
| PEG-100 | | | | 1 | 0.5 | | 2 | |
| stearate | | | | | | | | |
| Glyceryl | 2 | 3 | | | | | | |
| stearate (GMS) | | | | | | | | |
| Stearyl | | 1 | | | | | 5 | |
| alcohol | | | | | | | | |
| Cetearyl | | | 2 | | | 2 | | 1 |
| alcohol | | | | | | | | |
| Cetyl al- | | | | 1 | 1 | | | |
| cohol | | | | | | | | |
| Hydrogenated | 2 | | | 1 | | | | 1 |
| coconut fatty | | | | | | | | |
| glycerides | | | | | | | | |
| Shea butter | | 2 | | | | | | |
| C12-15 | | 3 | 3 | | 2 | 5 | 2 | |
| Alkyl | | | | | | | | |
| benzoate | | | | | | | | |
| Butylene | 1 | | | | | | | 2 |
| glycol di- | | | | | | | | |
| caprylate/ | | | | | | | | |
| dicaprate | | | | | | | | |
| Caprylic/ | | 4 | | | 1 | | | |
| capric tri- | | | | | | | | |
| glyceride | | | | | | | | |
| Hydrogenated | | | | | | | 1 | |
| polydecene | | | | | | | | |
| Ethylhexyl | 3 | | | | | | | 2 |
| coconut | | | | | | | | |
| fatty acid | | | | | | | | |
| ester | | | | | | | | |
| Octyldodec- | | | | | | 1 | | |
| anol | | | | | | | | |
| Mineral oil | | 1 | | | | 1 | | |
| Vaseline | 4 | | | 2 | | | | |
| Octa- | | 1 | 3 | 1 | 3 | 2 | | |
| methyltetra- | | | | | | | | |
| siloxane | | | | | | | | |
| Dimethyl- | | | 1 | | | | | 1 |
| polysiloxane | | | | | | | | |
| Dicaprylyl | 1 | 4 | | | | | | |
| ether | | | | | | | | |
| Dicarprylyl | | | | | 2 | | | 4 |
| carbonate | | | | | | | | |
| Polydecene | | | | 1 | | | 5 | |
| TiO₂ | | | 1 | 1 | | | 2 | 1 |
| Ethylhexyl | 3 | 2 | | 5 | 3 | | | 3 |
| methoxy- | | | | | | | | |
| cinnamate | | | | | | | | |
| 2-Ethylhexyl | | | 5 | | | | | |
| 2-cyano-3- | | | | | | | | |
| diphenyl- | | | | | | | | |
| acrylate | | | | | | | | |
| Ethylhexyl- | | 2 | | | 2 | 3 | | |
| triazone | | | | | | | | |
| Butylmethoxy- | | 1 | | 1 | | | | |
| dibenzoyl- | | | | | | | | |
| methane | | | | | | | | |
| Bis-ethyl- | 1 | | | | 1 | | | 2 |
| hexyloxy- | | | | | | | | |
| phenol- | | | | | | | | |
| methoxy- | | | | | | | | |
| phenyltri- | | | | | | | | |
| azines | | | | | | | | |
| Ubiquinone | 0.05 | | 0.05 | | 0.02 | | | 0.1 |
| (Q10) | | | | | | | | |
| Retinyl | | | | | | | 0.1 | |
| palmitate | | | | | | | | |
| Tocopheryl | | 0.5 | | | | | | |
| acetate | | | | | | | | |
| α-Gluco- | | | | | | 0.1 | | |
| sylrutin | | | | | | | | |
| Ascorbic acid | | | | 0.2 | | | | |
| Liponic acid | | | 0.1 | | | | | |
| Retinol | | | | | 0.1 | | | |
| Iminodi- | | 0.2 | | | | | | |
| succinic acid | | | | | | | | |
| Tetrasodium | 0.1 | — | 0.3 | 0.1 | 0.1 | 0.2 | 0.5 | 0.25 |
| iminodi- | | | | | | | | |
| succinate | | | | | | | | |
| Glycerin | 0.7 | 18 | 4 | 2 | 8 | 12 | 6 | 10 |
| Preserva- | q.s. | q.s. | q.s. | q.s. | q.s. | q.s. | q.s. | q.s. |
| tive, perfume, | | | | | | | | |
| thickener, | | | | | | | | |
| pH adjust- | | | | | | | | |
| ment and | | | | | | | | |
| solubilizing | | | | | | | | |
| agent | | | | | | | | |
| Water | to | to | to | to | to | to | to | to |
| | 100 | 100 | 100 | 100 | 100 | 100 | 100 | 100 |

That Which is claimed:

1. A cosmetic or dermatological topical formulation based on an oil-in-water emulsion, comprising
(a) 0.05 wt. % to 2 wt. %, based on the total weight of the formulation, of one or more ethoxylated fatty acid esters selected from the group consisting of PEG-5 to PEG-100 stearates;
(b) 0.5 wt. % to 20 wt. %, based on the total weight of the formulation, of glycerin; and
(c) one or more of:
(i) 0.1 wt. % to 6 wt. %, based on the total weight of the formulation, of glycerol monostearate, and
(ii) 0.1 wt. % to 8 wt. %, based on the total weight of the formulation, of one or more C₁₆-C₁₈ fatty alcohols.

2. The emulsion as claimed in claim 1, comprising both (c)(i) and (c)(ii).

3. The emulsion as claimed in claim 1, further comprising 0.1 wt. % to 30 wt. %, based on the total weight of the formulation, of one or more lipids having a polarity index of 5-30 mN/m.

4. The emulsion as claimed in claim 3, wherein the one or more lipids are present in an amount from 0.5 wt. % to 20 wt. %.

5. The emulsion as claimed in claim 1, further comprising 0.1 wt. % to 30 wt. %, based on the total weight of the formulation, of one or more lipids having a polarity index of 10-25 mN/m.

6. The emulsion as claimed in claim 5, wherein the one or more lipids are present in an amount from 0.5 wt. % to 20 wt. %.

7. The emulsion as claimed in claim 1, further comprising one or more additional compounds selected from the group consisting of active compounds, auxiliaries and additives.

8. The emulsion as claimed in claim 1, wherein (a) is present in an amount from 0.2 wt. % to 1 wt. %, based on the total weight of the formulation.

9. The emulsion as claimed in claim 1, wherein the ethoxylated fatty acid esters in (a) are selected from the group consisting of PEG-20 to PEG-60 stearates.

10. The emulsion as claimed in claim 1, wherein (b) is present in an amount from 1 wt. % to 10 wt. %, based on the total weight of the formulation.

11. The emulsion as claimed in claim 1, wherein (c)(i) is present in an amount from 0.5 wt. % to 3 wt. %, based on the total weight of the formulation.

12. The emulsion as claimed in claim 1, wherein (c)(ii) is present in an amount from 0.5 wt. % to 4 wt. %, based on the total weight of the formulation.

13. The emulsion as claimed in claim 1, further comprising coenzyme Q10.

14. The emulsion as claimed in claim 1, further comprising alpha-glucosylrutin.

15. The emulsion as claimed in claim 1, further comprising retinol.

16. A cosmetic or dermatological topical formulation based on an oil-in-water emulsion, comprising
(a) 0.2 wt. % to 1 wt. %, based on the total weight of the formulation, of one or more ethoxylated fatty acid esters selected from the group consisting of PEG-20 to PEG-60 stearates;
(b) 1 wt. % to 10 wt. %, based on the total weight of the formulation, of glycerin;
(c) one or more of:
(i) 0.5 wt. % to 3 wt. %, based on the total weight of the formulation, of glycerol monostearate, and
(ii) 0.5 wt. % to 4 wt. %, based on the total weight of the formulation, of one or more C₁₆-C₁₈ fatty alcohols; and
(d) 0.5 wt. % to 20 wt. %, based on the total weight of the formulation, of one or more lipids having a polarity index of 5-30 mN/m.

17. A method for moisturizing the skin, comprising applying to the skin a cosmetic or dermatological topical formulation based on an oil-in-water emulsion, comprising:
(a) 0.05 wt. % to 2 wt. %, based on the total weight of the formulation, of one or more ethoxylated fatty acid esters selected from the group consisting of PEG-5 to PEG-100 stearates;
(b) 0.5 wt. % to 20 wt. %, based on the total weight of the formulation, of glycerin; and
(c) one or more of:
(i) 0.1 wt. % to 6 wt. %, based on the total weight of the formulation, of glycerol monostearate, and
(ii) 0.1 wt. % to 8 wt. %, based on the total weight of the formulation, of one or more C₁₆-C₁₈ fatty alcohols.

18. The method as claimed in claim 17, wherein the emulsion comprises both (c)(i) and (c)(ii).

19. The method as claimed in claim 17, wherein (a) is present in an amount from 0.2 wt. % to 1 wt. %, based on the total weight of the formulation.

20. The method as claimed in claim 17, wherein the ethoxylated fatty acid esters in (a) are selected from the group consisting of PEG-20 to PEG-60 stearates.

21. The method as claimed in claim 17, wherein (b) is present in an amount from 1 wt. % to 10 wt. %, based on the total weight of the formulation.

22. The method as claimed in claim 17, wherein (c)(i) is present in an amount from 0.5 wt. % to 3 wt. %, based on the total weight of the formulation.

23. The method as claimed in claim 17, wherein (c)(ii) is present in an amount from 0.5 wt. % to 4 wt. %, based on the total weight of the formulation.

24. The method as claimed in claim 17, wherein the emulsion further comprises coenzyme Q10.

25. The method as claimed in claim 17, wherein the emulsion further comprises alpha-glucosylrutin.

26. The method as claimed in claim 17, wherein the emulsion further comprises retinol.

27. The method as claimed in claim 17, wherein the emulsion comprises:
(a) 0.2 wt. % to 1 wt. %, based on the total weight of the formulation, of one or more ethoxylated fatty acid esters selected from the group consisting of PEG-20 to PEG-60 stearates;
(b) 1 wt. % to 10 wt. %, based on the total weight of the formulation, of glycerin;
(c) one or more of:
(i) 0.5 wt. % to 3 wt. %, based on the total weight of the formulation, of glycerol monostearate, and
(ii) 0.5 wt. % to 4 wt. %, based on the total weight of the formulation, of one or more C₁₆-C₁₈ fatty alcohols; and
(d) 0.5 wt. % to 20 wt. %, based on the total weight of the formulation, of one or more lipids having a polarity index of 5-30 mN/m.

28. A method for one or more of limiting aging of the skin, limiting the formation of wrinkles, treating aged skin and treating wrinkles, comprising applying to the skin a cosmetic or dermatological topical formulation based on an oil-in-water emulsion, comprising:
(a) 0.05 wt. % to 2 wt. %, based on the total weight of the formulation, of one or more ethoxylated fatty acid esters selected from the group consisting of PEG-5 to PEG-100 stearates;
(b) 0.5 wt. % to 20 wt. %, based on the total weight of the formulation, of glycerin;
(c) one or more of:
(i) 0.1 wt. % to 6 wt. %, based on the total weight of the formulation, of glycerol monostearate, and
(ii) 0.1 wt. % to 8 wt. %, based on the total weight of the formulation, of one or more C₁₆-C₁₈ fatty alcohols; and
(d) one or more active ingredients selected from the group consisting of coenzyme Q10, alpha-glucosylrutin and retinol.
